(19) United States
(12) Patent Application Publication
(10) Pub. No.: US 2013/0123855 A1
(43) Pub. Date: May 16, 2013

(54) SPINAL COLUMN IMPLANT WITH PEDICLE SCREWS, AND THE CORRESPONDING PEDICLE SCREW

(71) Applicants: Jonathan Clark, Kloten (CH); Reto Braunschweiler, Neftenbach (CH); Thomas Zehnder, Bach (CH)

(72) Inventors: Jonathan Clark, Kloten (CH); Reto Braunschweiler, Neftenbach (CH); Thomas Zehnder, Bach (CH)

(73) Assignee: SPINELAB AG, Winterthur (CH)

(21) Appl. No.: 13/810,057

(22) PCT Filed: Jul. 5, 2011
(86) PCT No.: PCT/EP2011/061291

§ 371 Date: Jan. 14, 2013

(30) Foreign Application Priority Data
Jul. 12, 2010 (CH) 1127/10

(51) Int. Cl. A61B 17/70

(52) U.S. Cl. CPC A61B 17/7001
USPC 606/264

(57) ABSTRACT

A spinal column implant, comprising a connection element (9), and a plurality of pedicle screws (1) which are able to be screwed into the vertebral bodies of the spinal column and which are each provided with a head part (3) for receiving the connection element (9), with which a firm connection is able to be achieved between the pedicle screws (1) and the connection element (9), the connection element (9) being able to be assembled from rigid sections (6) and elastic sections (4), the rigid sections (6) being connectible to the elastic sections (4), wherein the rigid sections (6) and the elastic sections (4) of the connection element (9) are designed with a mutual form fit, and the head part (3) of the pedicle screws (1) is designed in such a way that the connection point of the rigid sections (6) and of the elastic sections (4) of the connection element (9) are firmly connectible, the rigid sections (6) as well as the elastic sections (4) each being able to be securely held by the head part (3) of the pedicle screw.

### TECHNICAL FIELD OF THE INVENTION

The present invention relates to a spinal column implant with pedicle screws and the corresponding pedicle screw.

Such a spinal column implant typically comprises a plurality of pedicle screws, which are screwable into the vertebral bodies of the spinal column, as well as a connection element, which is composed in each case of rigid sections and elastic sections. The pedicle screws are each provided with a head part for receiving the connection element, so that a firm connection is achievable between the pedicle screws and the connection element.

The advantage of such combined spinal column implants over implants having only rigid or only elastic connection elements is in particular that a rigid stabilization of the vertebral bodies can be achieved regionally while in the adjacent region the vertebral bodies are supported and stabilized with stiffening by an elastic system.

State of the Art

Spinal column implants of this kind are known from the state of the art. Disclosed in FR-A-2 844 180, for example, is a system in which elastic and rigid stabilization regions are used. Used thereby as elastic stabilization element is a spiral spring-plastic combination. This elastic stabilization element is connected to the screws via a pure clamping connection.

Another similar spinal column implant is also described in the document EP-A-1 815 812. Therein the surfaces of the elastic sections are provided with circumferential ribs and grooves, connection devices being provided for connection of rigid and elastic sections, which connection devices are made up of a U-shaped clamp made of a rigid material, having ribs and grooves at the bottom of the clamp and along the inner side of the arms, corresponding to the ribs and grooves of the elastic sections.

Disclosure of Invention

The object of the present invention now consists in creating a spinal column implant which continues to have the above-described advantages of combined spinal column implants, but however offers in addition a greater flexibility and more diverse possibilities of application. In addition a pedicle screw should be made available which can be used both within the framework of this combined spinal column implant as well as within the framework of other spinal column implants, i.e. also those with completely rigid or those with completely elastic connection elements. Also in general a spinal column implant should be created that gets by with as few elements as possible without losing in quality.

This object is achieved according to the invention in that, with a spinal column implant comprising a connection element and a plurality of pedicle screws that are screwable into the vertebral bodies and that are each provided with a head part for receiving the connection element, with which a firm connection is achievable between the pedicle screws and the connection element, the connection element being able to be assembled from rigid sections and elastic sections, the rigid sections being connectible to the elastic sections, the rigid sections and the elastic sections of the connection element are designed in a way formfitting with respect to one another, and that the head part of the pedicle screws is designed in such a way that the connection point of the rigid sections and of the elastic sections of the connection element is firmly connectible, both the rigid sections and also the elastic sections being each able to be firmly held by the head part of the pedicle screw.

With this design of the invention, not only is a modular system created in which the possibility exists of stabilizing in a rigid way vertebral bodies adjacent to each while other adjacent vertebral bodies are elastically supported and stabilized, whereby a limited possibility of movement is permitted, but also such a system is created having a much greater flexibility compared with previous systems.

In that the rigid sections of the connection element are designed in a formfitting way with respect to the elastic sections, a much better and much more secure connection of these elements can be ensured. Moreover the connection takes place in the region of the pedicle screw, so that supplementary devices for connection of the rigid sections to the elastic sections are not necessary. Thanks to the structured surfaces of the connection element, a good formfitting connection to the head part of the pedicle screw can be achieved. Since, in addition, no special connection elements are necessary, a maximal length of the elastic section can be achieved. This is extremely advantageous for the load of the elastic section, in particular with a minimal spacing between the pedicle screws.

Accordingly the present object is achieved according to the invention also by a pedicle screw which is screwable into the vertebral bodies of the spinal column and which is provided with a head part for receiving the connection element with which head part a firm connection between the pedicle screws and the connection element is achievable, the connection element being able to be assembled from rigid sections and/or elastic sections, the rigid sections being connectible to the elastic sections, the head part of the pedicle screw being designed in such a way that the connection point of the rigid sections and of the elastic sections of the connection element is firmly connectible, both the rigid sections as well as the elastic sections being in each case able to be firmly held by the head part of the pedicle screw.

Accordingly such a pedicle screw can be used both in connection with a connection element that is made up of rigid sections and elastic sections as well as also in connection with a connection element that is made up exclusively of elastic sections. Moreover no special connecting devices are necessary in order to hold together the individual sections.

One advantageous embodiment of the invention consists in that the pedicle screws are designed in such a way that the rigid sections and the elastic sections, respectively connected together, are aligned substantially coaxially. This results in great advantages and simplifications in the insertion of implants of this kind assembled from rigid and elastic sections.

In another advantageous embodiment of the invention, the head part of the pedicle screws is made up of a U-shaped clamp and a closure piece, between which the connection element is firmly clampable. A simple use of the spinal column implant can thereby be ensured.

Preferably the rigid sections are made of a metallic alloy, in particular of a titanium alloy, which leads to an optimal tolerance in the patients provided with such implants.

In an embodiment of the invention, the surfaces of the elastic sections are provided with circumferential ribs and grooves. A good formfitting connection to the pedicle screw without change of shape of the elastic section can thereby be ensured.

In another embodiment of the invention, the surfaces of the elastic sections are smooth. In this case the connection is given by a form fit brought about by force.

Preferably the elastic sections are made of a polyurethane-based biocompatible synthetic material. Also in this way an optimal tolerance is ensured.

Preferably the U-shaped clamp with the inserted elastic section is able to be closed off with a closure piece that is insertable between the clamp and is held via engagement means. A very simple procedure for the insertion and firm holding of the elastic section in the U-shaped clamp is thereby obtained, which can have positive consequences for the duration of the intervention.

A further advantageous embodiment of the invention consists in that the closure piece, in the state of being inserted in the U-shaped clamp, has on the side turned toward the section ribs and grooves corresponding to the ribs and grooves of the elastic section. The closure piece thereby also contributes in an optimal way to the formfitting connection between the elastic section and the U-shaped clamp.

Alternatively, to achieve a formfitting connection between the elastic section of the connection element and the closure piece, instead of the ribs and grooves, partial ribs or respectively partial ribs <sic. partial grooves> (i.e. ribs and grooves running over only a portion), nubs (e.g. in the shape of semicircular protuberances) or pins (e.g. in the shape of cylinders with a semicircular or similar end) can be used.

Preferably the connections devices are each attached at one end of a rigid section or on the head part of a screw. Resulting from this is an optimal disposition of these connection elements; the connection of the different sections to one another can take place in an optimal way.

Another advantageous embodiment of the invention consists in that the head parts of the screws are separated from the screws and have a receiving region for receiving a screw, the screw being connectible to the head part of the screw via a clamping screw with which the rigid section is able to be fixed in the head part of the screw. The screws can thereby be inserted first into the vertebral bodies, and the head part, in which the sections are able to be held, can be subsequently aligned with respect to the screw and the connection element.

Preferably, for receiving a screw, the receiving region of the head part of the screw has the shape of a spherical part, and the upper part of the screw is designed as spherical head. The head part thereby allows itself to be aligned optimally with respect to the screw and to be adjusted to the sections to be received therein.

### BRIEF DESCRIPTION OF THE DRAWINGS

Embodiments of the invention will be explained more closely in the following, by way of example, with reference to the attached drawing.

Shown are:

FIG. 1, a perspectival view of a spinal column implant according to one embodiment of the invention with a connection element which is assembled from a rigid section and an elastic section;

FIG. 2, a frontal view of the spinal column implant of FIG. 1 according to the invention;

FIG. 3, a perspectival sectional representation of the spinal column implant according to another embodiment of the invention;

FIG. 4, a schematic sectional representation of the upper region of a pedicle screw according to the invention with the connection region between the rigid section and the elastic section of the connection element; and

FIG. 5, a schematic sectional representation of the spinal column implant according to the invention with the connection region between the rigid section and the elastic section of the connection element.

### DETAILED DESCRIPTION OF THE PREFERRED EMBODIMENTS

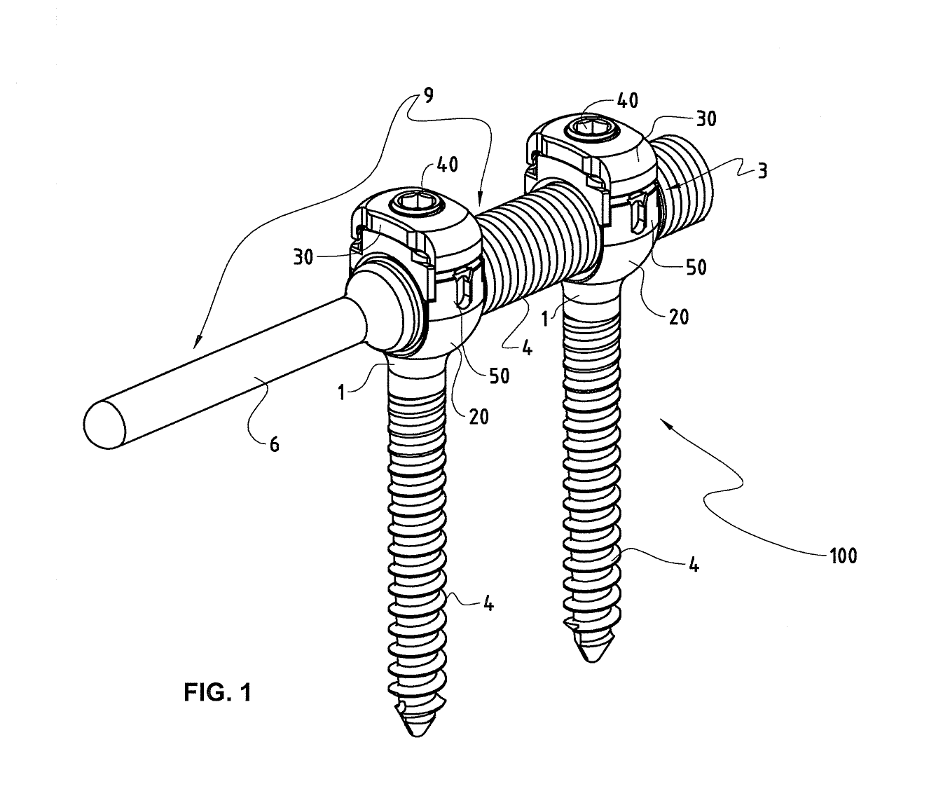

FIG. 1 shows a spinal column implant 100 according to the invention with a connection element 9, which is assembled from a rigid section 6 and from an elastic section 4. The connection element 9 is fixed by pedicle screws 1. It should be noted here however that spinal column implants can also be foreseen in which the connection element is assembled from elastic sections only.

These pedicle screws 1 are made up substantially of a screw body 4 and a head part 3. By means of the screw body 4, each pedicle screw 1 is screwed in each case into a vertebral body (not shown), so that a stabilization of the vertebral column can be brought about. The head part 3 of the pedicle screw 1 is designed in such a way that it is able to receive and fix the connection element 9. This receiving and fixing of the connection element will be described in detail later. The head part 3 is made up substantially of a U-shaped clamp 20 and an upper part 50 as well as a closure piece 30 and a corresponding clamping screw 40. Admittedly pedicle screws 1 are also conceivable in which the head part 3 also has other components.

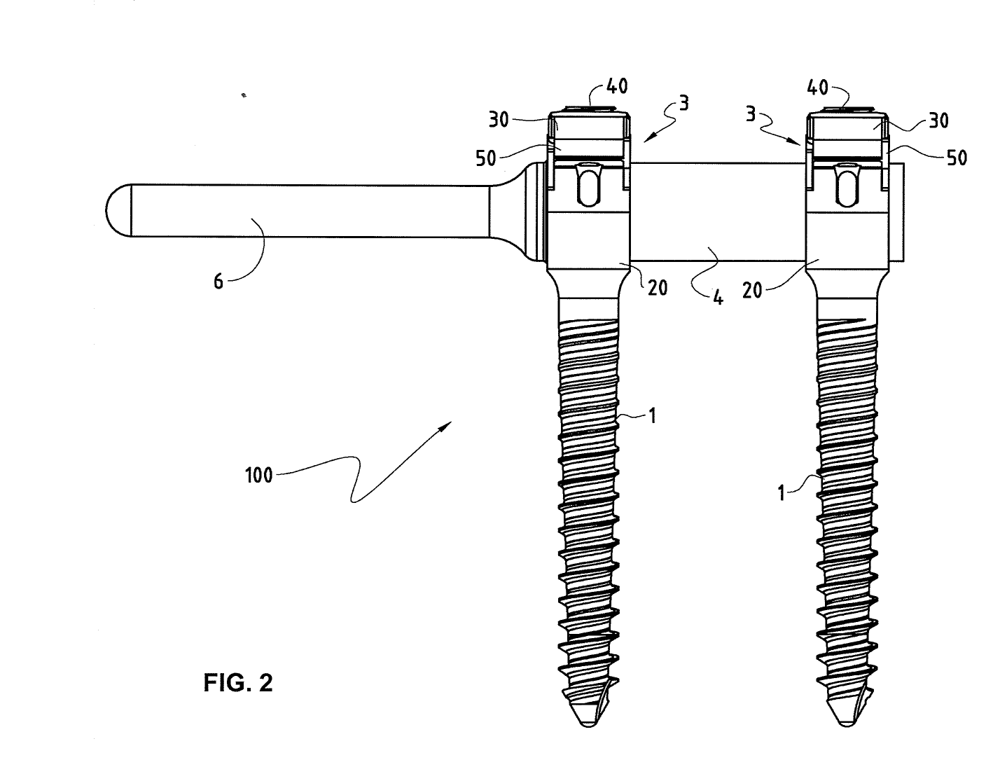

In FIG. 2, the spinal column implant according to the invention from FIG. 1 is shown in a frontal view. The same elements bear the same reference numerals. Thus it can be seen in FIG. 2 that the two sections of the connection element 9, namely the rigid section 6 and the elastic section 4, are connected to one another in such a way that their connection point is firmly connected by the head part 3 of the screw 1, both the rigid section 6 as well as the elastic section 4 being firmly held by the head part 3 of the pedicle screw 1. In this embodiment of the invention, the surface of the elastic section 4 is smooth and a form fit is achieved in a force-induced way with change of shape of the surface. This will be described in somewhat more detail later.

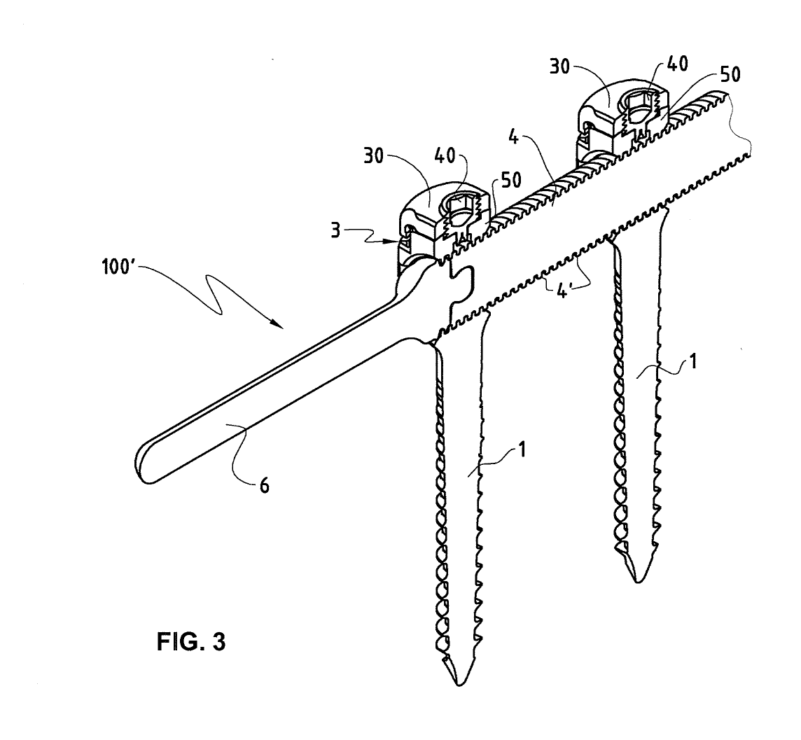

FIG. 3 shows the spinal column implant 100′ according to another embodiment of the invention in a perspectival sectional representation. In this spinal column implant 100′, the surface of the elastic section 4 of the connection element 9 is no longer smooth, but has ribs and grooves 4′. As can be seen in FIG. 3, a formfitting connection is produced between the ribs and grooves 4′ on the surface of the elastic section 6 and the corresponding ribs and grooves in the upper part 50 and in the U-shaped clamp 20 of the head part 3. This connection is then fixed by the closure piece 30 and the clamping screw 40.

One also sees in FIG. 3 (even if only rudimentarily—detailed explanation follows later), that the rigid section 6 of the connection element 9 also has ribs and grooves on its end piece, which ribs and grooves, in an analogous way, lead to a formfitting connection with the ribs and grooves of the upper part 50 and of the U-shaped clamp 20. This connection is also additionally clamped by the closure piece 30 and the clamping screw 40.

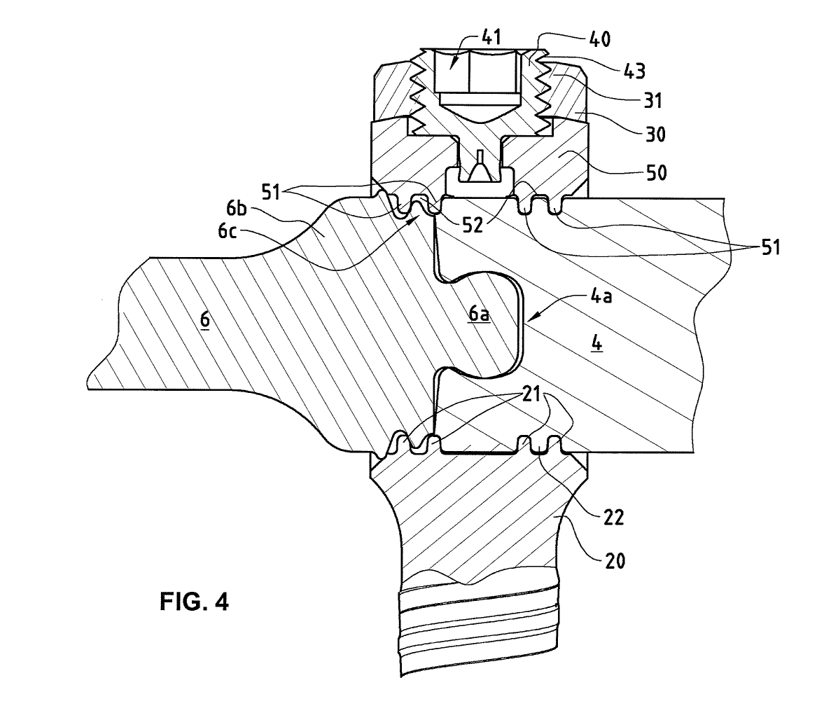

The structure of the head part 3 of a pedicle screw 1 according to the invention is shown in detail in FIG. 4. It is clearly shown how the rigid section 6 is connected to the elastic section 4 by means of a formfitting connection. In an exemplary way, a groove and plug connection is shown here. Of course other connection possibilities could also be used, however.

The rigid section 6 thus comprises a projection 6a, which forms a formfitting connection with the corresponding hollow 4a in the elastic section 4. Of course it is conceivable to design the connection mirror-inverted, so that the projection comes to be situated on the elastic section and the hollow on the rigid section.

This connection point is firmly connected by way of the head part 3 of the pedicle screw 1. For this purpose this connection point is clamped between the U-shaped clamp 20 and the upper part 50. The closure piece 30 with the clamping screw 40 clamp these two elements together so that a firm connection is made. For this purpose the clamping screw 40 comprises a hole 41, thanks to which this screw 40 is able to be tightened by a corresponding tool. Provided on the outer edge of the clamping screw 40 is a male thread 43, which is able to assume a firm connection with the matchable female thread 31 of the closure piece 30. Upon tightening of the clamping screw 40, the upper part 30 <sic. 50> is pressed downward whereby the connection point of the elastic section 4 with the rigid section 6 is firmly clamped by the upper part 50 and the U-shaped clamp 20.

As can be seen in FIG. 4, the U-shaped clamp 20 has ribs 21 and grooves 22, while the upper part 50 has ribs 51 and grooves 52. These ribs 21, 51 and grooves 22, 52 correspond to the ribs and grooves 6c, which are formed on the rigid section 6. Upon tightening of the clamping screw 40 a formfitting connection is likewise formed between these elements. Since a rigid section is moreover involved, this also serves as a stop for the tightening of the clamping screw 40. In this way an extremely stable attachment of the rigid section 6 to the pedicle screw 1 is able to be achieved.

According to the invention, a formfitting connection is likewise produced on the side of the elastic section 4. In FIG. 4 this is achieved in such a way that the ribs 21, 51 and grooves 22, 52 change the surface of the elastic element 4 during tightening of the clamping screw 40 until no movements between the elastic element 4, on the one hand, and the U-shaped clamp 20 and the upper part 50, on the other hand, are possible. Together with the form fit between the rigid section 4 <sic. 6> and the elastic section 6 _{<}sic. 4>, as well as the firm connection between the U-shaped clamp 20 and the upper part 50 with the rigid section 4 <sic. 6>, this combination results in a very stable fixed connection element 9.

If the surface of the elastic element 4 is not smooth, however, but also has ribs and grooves—as in FIG. 3, for example,—a formfitting connection is achieved between this elastic section 4 and the head part 3 of the screw 1 without change of shape of the surface of the elastic section 4. Thus the elastic sections 4 also behave the same way as the rigid sections 6 in such an embodiment.

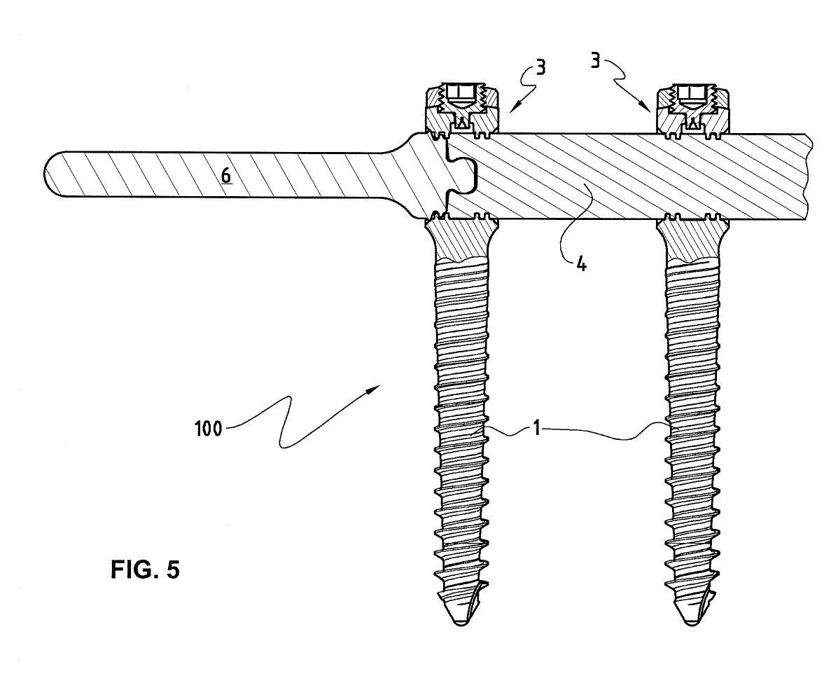

Finally, FIG. 5 shows a larger representation of the spinal column implant 100 according to the invention, in which the firm holding of the connection region between the rigid section 6 and the elastic section 4 of the connection element 9 by the head part 3 of the screw 1 is shown a little more clearly.

In closing, it is pointed out that the embodiment variants described here by way of example represent only a selection of possible implementations of the concepts according to the invention, and should in no way be seen as limiting. One skilled in the art will understand that many other implementations of the invention are possible without the essential features of the invention being lost sight of.

1. A spinal column implant, comprising a connection element, and a plurality of pedicle screws which are able to be screwed into the vertebral bodies of the spinal column and which are each provided with a head part for receiving the connection element, with which a firm connection is able to be achieved between the pedicle screws and the connection element, the connection element being able to be assembled from rigid sections and elastic sections, the rigid sections being connectible to the elastic sections,
wherein
the rigid sections and the elastic sections of the connection element are designed with a mutual form fit, and
the head part of the pedicle screws is designed in such a way that the connection point of the rigid sections and of the elastic sections of the connection element is firmly connectible, the rigid sections as well as the elastic sections each being able to be securely held by the head part of the pedicle screw.

2. The spinal column implant according to claim 1, wherein the elastic sections of the connection element are made of a biocompatible synthetic material.

3. The spinal column implant according to claim 1, wherein the surfaces of the elastic sections are provided with circumferential ribs and grooves.

4. The spinal column implant according to claim 1, wherein the surfaces of the elastic sections are smooth.

5. A pedicle screw, which is screwable into the vertebral bodies of the spinal column and which is provided with a head part for receiving the connection element, with which a firm connection is achievable between the pedicle screw and the connection element, the connection element being able to be assembled from rigid sections and elastic sections, the rigid sections being connectible to the elastic sections,
wherein
the head part of the pedicle screw is designed in such a way that the connecting point of the rigid sections and of the elastic sections of the connection element are firmly connectible, the rigid sections as well as the elastic sections being in each case able to be firmly held by the head part of the pedicle screw.
